(19) United States
(12) Patent Application Publication
(10) Pub. No.: US 2016/0022254 A1
(43) Pub. Date: Jan. 28, 2016

(54) DEVICES AND METHODS FOR FIXATION OF ANIMAL TISSUE

(71) Applicant: Boston Scientific Scimed, Inc., Maple Grove, MN (US)

(72) Inventor: Ray McCarthy, Galway (IE)

(21) Appl. No.: 14/804,956

(22) Filed: Jul. 21, 2015

Related U.S. Application Data
(60) Provisional application No. 62/027,926, filed on Jul. 23, 2014.

(51) Int. Cl. A61B 17/02

(52) U.S. Cl. CPC A61B 17/02; A61B 2017/0287; A61B 2017/0237

(57) ABSTRACT

This document relates to tissue modification and, more particularly, to modification of biological tissue for implantation in a mammal. For example, this document relates to tissue tensioning devices and methods used during fixation of tissues for bioprosthetic heart valves.

### CLAIM OF PRIORITY

This application claims priority to U.S. Provisional Application Ser. No. 62/027,926, filed on Jul. 23, 2014, the entire contents of which are hereby incorporated by reference.

### BACKGROUND

1. Technical Field

This document relates to tissue modification and, more particularly, to modification of biological tissue for implantation in a mammal. For example, this document relates to devices and methods used during fixation of tissues used in bioprosthetic heart valves.

2. Background Information

Heart valve surgery can be used to repair or replace diseased heart valves. For example, heart valve replacement may be indicated when there is a narrowing of the native heart valve, commonly referred to as stenosis, or when the native valve leaks or regurgitates. The repair or replacement of diseased heart valves can include, for example, the introduction of a prosthetic heart valve that includes biological tissue heterologous to the patient (e.g., a heterograft or xenograft).

Biological tissue can have mechanical properties that vary within a single donor and/or from among several donors of the same species. For example, biological tissue from a single donor can have non-uniform thickness, and the average thickness of biological tissue can vary from one donor to another. The variation in mechanical properties of biological tissue used in replacement heart valves can impact the performance and/or durability of a replacement heart valve implanted in a patient.

### SUMMARY

This document provides devices and methods for tissue modification and, more particularly, to modification of biological tissue for implantation in a mammal. For example, this document provides devices and methods used during fixation of tissues for bioprosthetic heart valves. The devices and methods provided herein can be used, for example, to maximize the usable area of the harvested biological tissue and to control the tension of the tissue during fixation.

Pericardial tissue from the sacs surrounding the hearts of animals, e.g. bovine, porcine, and equine, etc., are used to create tissues that can be used in various medical applications. Bioprosthetic heart valves are one such application. The pericardial sacs are chemically treated to reduce the antigenicity of the xeno-tissue and to crosslink the collagen fibers, thereby resulting in a fixed tissue biomaterial that resists the host's immune response and has the mechanical properties required to perform as a replacement heart valve.

In one implementation, a tissue tensioning apparatus for tensioning a tissue includes a peripheral frame defining an interior space; a plurality of elongate tensioning members each including a tissue attachment member configured to attach to the tissue, each of the plurality of tensioning members being attachable to the frame such that the tissue attachment member is disposed within the interior space; and a frame size adjustment feature coupled to the frame and arranged to adjust a configuration of the frame and positions of the tissue attachment members relative to the interior space when the plurality of tensioning members are attached to the frame such that the tissue attachment members are disposed within the interior space.

Such a tissue tensioning apparatus may optionally include one or more of the following features. The interior space may be circular. The interior space may be generally triangular. The interior space may be polygonal. The tissue attachment member may comprise a clip. The tissue attachment member may comprise a hook. The frame size adjustment feature may comprise a rotatable screw or worm gear that meshes with features on the frame. The frame size adjustment feature may comprise an inflatable balloon member. The frame size adjustment feature may comprise a pivot arm. The frame size adjustment feature may be configured to bi-axially adjust the positions of the tissue attachment members relative to the interior space. The frame size adjustment feature may be configured to adjust the positions of the tissue attachment members relative to the interior space in four or more different directions. The frame size adjustment feature may be configured to adjust the positions of the tissue attachment members relative to the interior space in eight or more different directions. The frame size adjustment feature may be configured to adjust the positions of the tissue attachment members relative to the interior space in twelve or more different directions. The frame may include a plurality attachment features whereby the plurality of elongate tensioning members are attachable to the frame. The plurality of attachment features may comprise slots. The plurality of attachment features may comprise clamps.

In another implementation, a method for tissue modification includes attaching a tissue to a tissue tensioning apparatus and adjusting the frame size adjustment feature to apply tension to the tissue. The tissue tensioning apparatus includes a peripheral frame defining an interior space; a plurality of elongate tensioning members each including a tissue attachment member configured to attach to the tissue, each of the plurality of tensioning members being attachable to the frame such that the tissue attachment member is disposed within the interior space; and a frame size adjustment feature coupled to the frame and arranged to adjust a configuration of the frame and positions of the tissue attachment members relative to the interior space when the plurality of tensioning members are attached to the frame such that the tissue attachment members are disposed within the interior space.

Such a method for tissue medication can optionally include one or more of the following features. An entirety of the tissue may be within the interior space when the tissue is attached to the tissue tensioning apparatus.

Particular embodiments of the subject matter described in this document can be implemented to realize one or more of the following advantages. First, in some embodiments the frames of the tissue tensioning devices provided herein are larger than the outer periphery of the tissue sample mounted thereto. Since the frame is larger than the tissue, all of the tissue can be tensioned and further processed. That is, none of the tissue is trimmed away or not treated by tensioning during the fixation process. As a result, all of the tissue can be processed and potentially used in prosthetic devices. In some implementations, animal pericardial tissue is used in conjunction with the devices and methods provided herein. The pericardial tissue may have different thicknesses at different locations of the sample. Because the entire sample is processed, the tissues of different thicknesses can be advantageously used in differently sized prosthetic devices, such as differently sized heart valves.

Second, in some embodiments the devices and methods provided herein facilitate accurately controllable tensioning of the tissue sample. Therefore, desirable, predictable, and repeatable final results can be attained. More specifically, the tension of the tissue during the fixation process can affect one or more mechanical properties of the tissue. For example, in some circumstances the stiffness and/or elasticity of the final fixed tissue is affected by the tension of the tissue during the fixation process. Therefore, accurate control of the tension of the tissue during the fixation process can result in a final tissue product with the desired mechanical properties for a particular application.

Moreover, setting the tissue stress load in these embodiments can result in a set stress to the tissue and variable amount of strain applied to portions of the tissue. For example, pieces of tissue having different stress-strain characteristics will be stretched different distances under the same set stress load. In some example, setting the stress load and allowing the resulting strain in the tissue to vary can improve the uniformity of the mechanical properties (e.g., stiffness along a first axis and a second axis) across several pieces of tissue. This improved uniformity can facilitate tissue matching for leaflets used in a prosthetic heart valve, where matching mechanical properties of the tissue used for the leaflets can improve the load distribution over the leaflets and, thus, improve hemodynamic performance and/or improve the durability of the prosthetic heart valve. In a prosthetic heart valve including leaflets made from tissue, the improvements in the similarity of stiffness characteristics along the various axes of the tissue can result in improved load distribution and/or durability in the valve.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a flowchart of an example process for processing bovine pericardial tissue into a bioprosthetic heart valve.

FIG. 2A is a top view of an example tissue fixation device in accordance with some embodiments.

FIG. 2B is a partial cross-sectional side view of the tissue fixation device of FIG. 2A.

FIG. 3A is a top view of another example tissue fixation device in accordance with some embodiments.

FIGS. 3B and 3C are partial cross-sectional side views of the tissue fixation device of FIG. 3A.

FIG. 4A is a top view of another example tissue fixation device in accordance with some embodiments.

FIGS. 4B and 4C are partial cross-sectional side views of the tissue fixation device of FIG. 4A.

FIG. 5A is a top view of another example tissue fixation device in accordance with some embodiments.

FIGS. 5B and 5C are partial cross-sectional side views of the tissue fixation device of FIG. 5A.

FIG. 6 is a top view of another example of tissue fixation device in accordance with some embodiments.

Like reference numbers represent corresponding parts throughout.

### DETAILED DESCRIPTION

This document relates to tissue modification and, more particularly, to modification of biological tissue used for implantation in a mammal. For example, this document relates to devices used during fixation of tissues used in bioprosthetic heart valves. In some implementations, the tissue can be bovine pericardium, equine pericardium, porcine pericardium, and the like. In some implementations, the tissue is a patch cut from a pericardial sac. While this disclosure describes devices used during fixation of tissues used for bioprosthetic heart valves, the tissues produced by the devices disclosed herein may also be used in other applications such as, but not limited to, vascular grafts, ligament substitutes, pericardial patches, and the like.

Pericardial tissue from the sacs surrounding the hearts of animals, e.g. bovine, porcine, and equine, etc., are commonly used to create tissues that can be used in various medical applications. Bioprosthetic heart valves, such as Boston Scientific's Lotus™ Valve, are one such application. The pericardial sacs are chemically treated to reduce the antigenicity of the xeno-tissue and to crosslink the collagen fibers, thereby resulting in a fixed tissue biomaterial that resists the host's immune response and has the mechanical properties required to perform as a replacement heart valve. This disclosure describes apparatuses and methods to be used during the tissue fixation process.

Most current fixation procedures involve the use of a buffered, aqueous solution with salts and glutaraldehyde. Glutaraldehyde, the active component in the fixing solution, has the dual role of reducing antigenicity of the tissue and cross-linking the proteinaceous collagen. The glutaraldehyde concentration is usually in the range of 0.2 to 0.7%. The cross-linking process ‘locks in’ the mechanical properties of the tissue. As such, the degree of tension seen by the tissue during fixation has an effect on the mechanical properties of the final fixed tissue. For example, tissue fixed in a stretched state will tend to have less elasticity than tissue fixed non-tensioned. Likewise, tissue that is tensioned uniaxially will tend to have more elasticity in the direction perpendicular to the direction of tensioning. Biaxial tensioning will tend to result in a more uniform elastic modulus of the tissue.

For at least the foregoing reasons, the apparatus used to hold and tension the tissue during fixation plays a significant role in the final mechanical properties of the fixed tissue, and as a result the apparatus may also affect the performance of any bioprosthetic valve in which the tissue is used. Prior to fixation the pericardial sacs are cut open. Typically the sac is cut in half by making cuts from base to apex, resulting in two triangular shaped pieces of tissue. The thickness of the tissue varies, with the thickest tissue generally found at the base of the sac and the thinnest found at the apex of the sac.

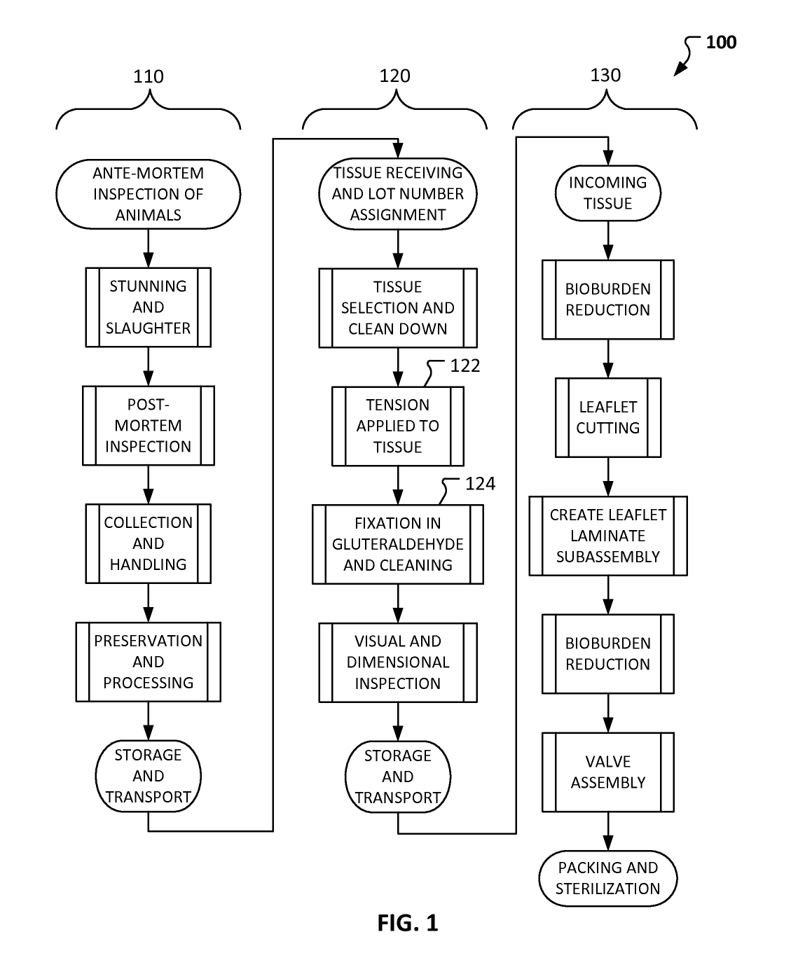

Referring to FIG. 1, an example method 100 for harvesting and processing tissue into a bioprosthetic heart valve includes a series of abattoir steps 110, a series of tissue fixation steps 120, and a series of prosthetic manufacturing steps 130. While the method 100 is described in the context of harvesting and processing bovine pericardium tissue to construct heart valves, these concepts can also be applied to obtaining and processing other types of biological tissue and/or for constructing other types of prostheses. Such other types of biological tissue can include, but are not limited to porcine, bovine, ovine, or other aortic or pulmonary valves and vascular tissues; human donor allografts; omentum or other tissues of the digestive tract; skin, placenta, uterus, or tissues reconstructed in vitro from cells from such tissues; and ocular tissues including cornea and sclera. It should be understood that the method 100 is merely one non-limiting example of a method for processing tissue into a bioprosthetic heart valve using the tissue tensioning apparatuses provided herein, and that the tissue tensioning apparatuses can also be used in the context of other methods for processing tissues.

In some implementations, the series of abattoir (slaughterhouse) steps 110 includes ante-mortem inspection of animals, stunning and slaughter, post-mortem inspection of the animals, collection and handling of the animal's pericardium, preservation and processing of the pericardium, and storage and transport of the pericardium. In these series of steps 110 the pericardium is removed from the heart, inspected, processed, preserved, prepared for shipment, and shipped. At various times the pericardium may be stored on ice. During processing, the fat pad is removed from the pericardium and the pericardium is rinsed one or more times (e.g., three times in a saline solution). The pericardium may be soaked in a tissue preservative solution. The soaking pericardium may be packed on ice and shipped to a facility for tissue fixation.

In some implementations, the series of tissue fixation steps 120 includes tissue incoming receiving and lot number assignment, tissue selection and clean down, tissue tensioning 122, fixation in glutaraldehyde 124, visual and dimensional inspection, and storage and transport. During the step of tissue tensioning 122, a tissue tensioning apparatus such as those described herein can be coupled to the pericardial tissue. In some implementations, the loads applied to the tissue by the tensioning are about 0.1 N to about 2 N. In certain implementations, the stress applied to the tissue is about 0.01 N/mm² to about 2 N/mm²However, other load and stress levels are also envisioned within the scope of this disclosure. With the pericardium tissue coupled to the tissue tensioning apparatus, the step of fixation in glutaraldehyde 124 can be performed.

In some implementations, the stress loads are applied to the tissue by the tissue tensioning apparatus for about 30 minutes to about 120 minutes (e.g., in the glutaraldehyde solution). In certain embodiments, the tissue can be removed from the tissue tensioning apparatus and exposed to a glutaraldehyde solution for about one day to about two weeks. Additionally or alternatively, the tissue can be mounted on the tissue tensioning apparatus and exposed to a non-cross linking-solution (e.g., phosphate-buffered saline or saline) for about 30 minutes to about 120 minutes prior to exposure to the glutaraldehyde solution for about one day to about two weeks.

In some implementations, the tissue is held in the stretched position such that the average thickness of the tissue is reduced. For example, the average thickness of the tissue held in the stretched position can be about 0.1 mm to about 0.4 mm. In some implementations, biaxial stretching of tissue and fixing the tissue results in little to no increase in thickness in the tissue. In these embodiments, as compared to fixing tissue under uniaxial stress loading or no stress loading, multi-axial stretching can result in thinner fixed tissue. In implementations in which the tissue is part of an intraluminally delivered prosthetic heart valve, such a reduction in the thickness of the tissue can, for example, reduce the sheathing forces associated with the prosthetic heart valve and/or reduce the overall profile of the prosthetic heart valve for easier delivery to the implantation site.

After fixation in glutaraldehyde, the pericardium is inspected, stored, and transported to a facility for prosthetic manufacturing.

In some implementations, the series of prosthetic manufacturing steps 130 includes one or more instances of bioburden reduction, leaflet cutting, creating leaflet laminate subassemblies, valve assembly, packaging, and sterilization. As an outcome of performing method 100, one or more bioprosthetic heart valves are created using bovine pericardium. The pericardial tissue of the valve's leaflets have mechanical properties resulting from the steps of applying tension to the pericardial tissue 122 and tissue fixation 124. The tissue tensioning apparatuses provided herein can be used during the steps of applying tension to the pericardial tissue 122 and tissue fixation 124.

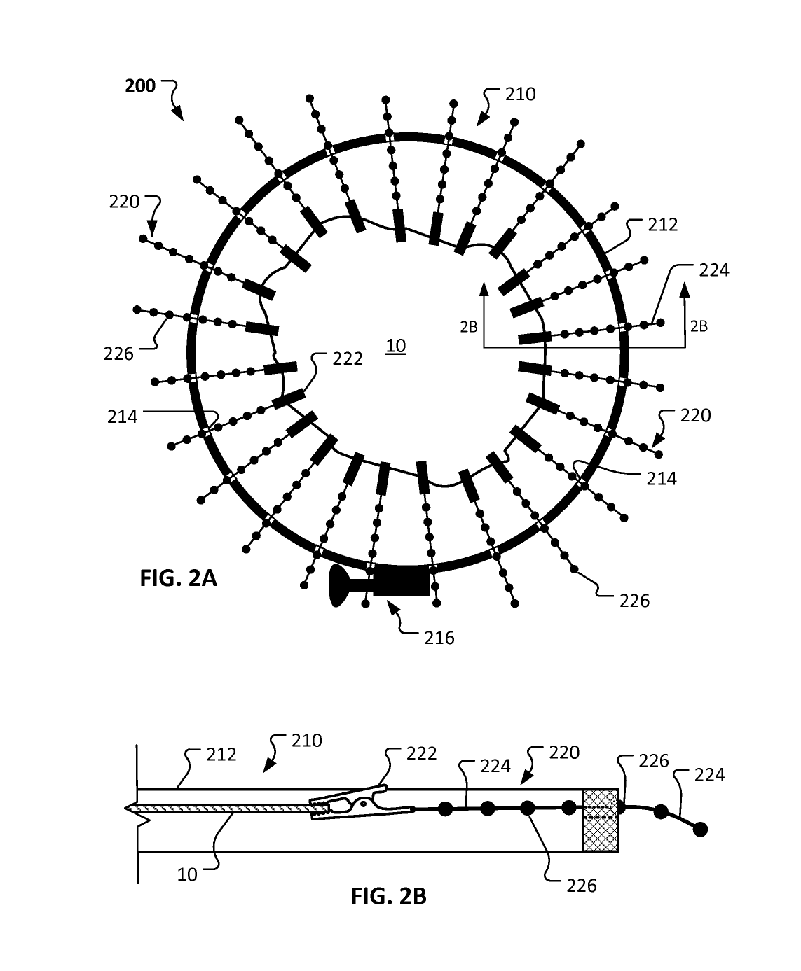

FIGS. 2A, 3A, 4A, 5A, and 6 provide examples of the tissue tensioning apparatuses provided herein. In general, the example tissue tensioning apparatuses include multiple tensioning members having a first end that couples with the tissue and a second end that couples with an outer frame. The tissue is thereby suspended by the tensioning members within an inner space defined by the outer frame.

In some embodiments, the outer frame is adapted to apply tension to the multiple tensioning members, which in turn apply tension to the tissue. For example, some tissue tensioning apparatuses provided herein have a frame that is mechanically expandable to apply tension to the multiple tensioning members, which in turn apply tension to the tissue. In other examples, the tissue tensioning apparatuses include a balloon (inflatable bladder member) on the frame. The balloon can be inflated to apply tension to the multiple tensioning members, which in turn apply tension to the tissue. It should be understood that other types of mechanisms can alternatively or additionally be included to apply tension to the multiple tensioning members, without departing from the scope of this disclosure.

The tissue tensioning apparatuses provided herein can be operated to apply tension to a tissue using the following general technique. It should be understood that this is just one example technique, and that other techniques can also be used without departing from the scope of this disclosure. A first tensioning member can be coupled to the tissue. The first tensioning member can then be coupled to the frame such that the tissue is nominally centered within the inner space defined by the frame. Next, a second tensioning member that is located approximately 180 degrees opposite from the first tensioning member can be coupled to the tissue. The second tensioning member can then be coupled to the frame. A third tensioning member can then be coupled to the tissue and to the frame. A fourth tensioning member that is approximately 180 degrees opposite from the third tensioning member can then be coupled to the tissue and to the frame. At this stage, the coupling of the tensioning members to the frame results in only a minimal amount of tension applied to the tissue. This process of coupling opposing tensioning members to the tissue and frame is repeated until all tensioning members are coupled to the tissue and to the frame. In some embodiments, about 20 to about 30 tensioning members are included.

However, in some embodiments fewer than 20 or more than 30 tensioning members are included. When all tensioning members are so coupled, the tissue is suspended by the tensioning members, but minimal tension (other than that which is required to suspend the tissue) is applied to the tissue. Thereafter, the frame can be expanded or otherwise adjusted to apply tension to the tissue in a controlled fashion.

In some embodiments, instrumentation for measuring and controllably applying tension to the tissue is included as part of the tissue tensioning apparatus. For example, in some embodiments one or more strain gauges are included on one or more of the tensioning members. In some embodiments, a torque measuring instrument is used when applying a torque to expand the frame. In some embodiments, an inflation media pressure control device and indicator is used to inflate a balloon that is on the frame and that applies tension to the multiple tensioning members. The type of frame used and the locations of the tensioning members used can also be selected to obtain a desired configuration of applied tension. For example, some frames and/or tensioning member patterns can be selected to assert uniaxial tension, biaxial tension, 360° tension, and other tension configurations as desired.

It should be understood that one or more features of one or more of the tissue tensioning apparatuses provided herein can be combined with one or more features of one or more other tissue tensioning apparatuses provided herein to create hybrid designs of the tissue tensioning apparatuses. Said another way, the features of the tissue apparatuses provided herein can be mixed and matched in all possible permutations and configurations as desired, and such resulting apparatuses are within the scope of this disclosure.

Referring to FIGS. 2A and 2B, an example tissue tensioning apparatus 200 uses a frame 210 and multiple tensioning members 220 to exert tensile forces to a tissue 10. The multiple tensioning members 220 suspend the tissue 10 within an inner space defined by the frame 210. The frame 210 can be expanded to apply tension to the multiple tensioning members 220, which in turn apply tension to the tissue 10.

In the depicted embodiment, the multiple tensioning members 220 each include a clip 222, an elongate element 224, and multiple gripping features 226. The clip 222 is coupled to one end of the elongate element 224. The multiple gripping features 226 are coupled to the elongate element 224 at spaced apart positions along the length of the elongate element 224.

In the depicted embodiment, the frame 210 includes a circular peripheral element 212 and a frame adjustment mechanism 216. In some embodiments, the frame 210 can have other shapes such as, but not limited to, rectangular, triangular, elliptical, and the like. In some embodiments, the diameter of the peripheral element 212 is about 300 mm to about 350 mm. However, peripheral elements 212 having sizes smaller than 300 mm or larger than 350 mm in diameter are also envisioned within the scope of this disclosure.

The frame adjustment mechanism 216 is coupled to the peripheral element 212 in an arrangement such that the frame adjustment mechanism 216 can be manipulated to increase and decrease the diameter of the peripheral element 212. In that sense, the frame adjustment mechanism 216 acts as a frame size adjustment feature. Other types of frame size adjustment features (inflatable members, mechanical linkages, etc.) can be alternatively or additionally included as part of the tissue tensioning apparatus 200. The peripheral element 212 includes multiple tension member engagement features 214 that are spaced apart from each other at positions on the peripheral element 212.

In the depicted embodiment, one end of each of the multiple tensioning members 220 is releasably coupled to the tissue 10 using the clip 222. A gripping feature 226 of each tensioning member 220 is releasably engaged with one of the tension member engagement features 214 located on the frame 210. In this arrangement, the multiple tensioning members 220 can suspend the tissue 10 within an inner space defined by the frame 210.

With the tissue 10 so suspended within an inner space defined by the frame 210, the frame adjustment mechanism 216 can be manipulated to increase and/or decrease the diameter of the frame 210. Increasing or decreasing the diameter of the frame 210 adjusts the tension of the multiple tensioning members 220 and the tissue 10. In particular, increasing the diameter of the frame 210 increases the tension of the multiple tensioning members 220 and the tissue 10, while decreasing the diameter of the frame 210 decreases the tension of the multiple tensioning members 220 and the tissue 10. Using this arrangement, the tension of individual tensioning members 220 are controlled and maintained substantially equal to each other.

In some embodiments, the peripheral element 212 of the frame 210 is metallic or at least partially metallic. For example, in some embodiments the peripheral element 212 comprises stainless steel (e.g., L605, 304, 304L, 316, etc.), alloy steels, aluminum, or aluminum alloys to provide a few examples. In some embodiments, the peripheral element 212 of the frame 210 is polymeric or at least partially polymeric. For example, in some embodiments the peripheral element 212 comprises polyamide, polyethylene, polytetrafluoroethylene, or polyvinyl chloride to provide a few examples. In some embodiments, the peripheral element 212 comprises a metallic portion and a polymeric portion. For example, in some such embodiments the tension member engagement features 214 may comprise a polymeric material while other portions of the frame 210 comprise one or more metallic materials.

In the depicted embodiment, the tension member engagement features 214 are slots in the peripheral element 212. The use of slots as the tension member engagement features 214 is just one type of tension member engagement feature 214 that can be used. In some embodiments, the tension member engagement features 214 can be clamps, screws, eyelets, receptacles, collets, wedges, cleats, chocks, and various other types of engagement features, and combinations thereof. The widths of the slots 214 are less than the size of the outer profile of the gripping features 226, resulting in an interference fit therebetween. Therefore, the engagement of the tension member engagement features 214 with the gripping features 226 fixes the tensioning members 220 to the frame 210, thereby allowing the tensioning members 220 to be tensioned when the frame 210 is expanded.

The frame 210 also includes the frame adjustment mechanism 216. The frame adjustment mechanism 216 is configured to expand and retract the diameter of the frame 210 by manipulation of the frame adjustment mechanism 216. Using this arrangement, the tension of individual tensioning members 220 are controlled substantially equal to each other. In the depicted embodiment, the frame adjustment mechanism 216 comprises a screw or worm gear that meshes with complementary features on the peripheral element 212 to establish a worm gear and worm wheel type of arrangement. The peripheral element 212 and frame adjustment mechanism 216 cooperate to increase or decrease the diameter of the peripheral element 212 in the manner of a hose clamp device. In some embodiments, the frame adjustment mechanism 216 may take other forms that serve to expand and retract the diameter of the frame 210 without departing from the scope of this disclosure.

In some embodiments, the torque to adjust the frame adjustment mechanism 216 is proportional to the tension applied to the tissue 10. Therefore, in some such embodiments a predetermined amount of torque may be applied to the frame adjustment mechanism 216 to result in a desired tensioning of the tissue 10. In some implementations, a torque measuring instrument may be used to apply the torque and to thereby attain a desired amount tension on the tissue 10.

The tissue tensioning apparatus 200 also includes the multiple tensioning members 220. The multiple tensioning members 220 each include the clip 222, the elongate element 224, and multiple gripping features 226.

In the depicted embodiment, the clip 222 is an alligator clip 222. In some embodiments, other types of devices and/or other techniques for coupling the tensioning members 220 to the tissue 10 are used. For example, in some embodiments, hooks, sutures, barbs, adhesives, clamps, and the like, and combinations thereof can be used to couple the tensioning members 220 to the tissue 10 are used.

Each of the multiple tensioning members 220 also includes the elongate element 224. In some embodiments, the elongate element 224 is a filament, a wire, a string, a shaft, a chain, and the like, and/or combinations thereof. The elongate element 224 can comprise metallic (e.g., stainless steels, alloy steels, nitinol, brass, etc.) or polymeric materials, or a combination thereof. In some embodiments, the elongate element 224 is flexible, but flexibility is not a requirement. In some embodiments, the elongate element 224 does not appreciably stretch when tension is applied to the elongate element 224 by the expansion of the frame 210. Alternatively, in some embodiments the elongate element 224 may stretch when so tensioned.

Each of the multiple tensioning members 220 also includes one or more gripping features 226. In embodiments having multiple gripping features 226, a particular gripping feature 226 of the multiple gripping features 226 can be selected to be engaged with a corresponding tension member engagement feature 214 of the frame 210. The selection of the particular gripping feature 226 can be determined so as to properly suspend the tissue 10 while imparting minimal additional tension thereto.

In some embodiments, the gripping features 226 are affixed to the elongate element 224. In some embodiments, the multiple gripping features 226 are movably fixable to the elongate element 224 (e.g., using a set screw, twist lock, compression fit, collet, clamp arrangement, etc.). In some embodiments, the gripping features 226 are equally spaced apart from each other at locations along the elongate element 224. In some embodiments, some of the spacing between the gripping features 226 varies or is variable along the elongate element 224 (e.g., using graduated spacing, etc.).

In the depicted embodiment, the gripping features 226 are spherical and on center with the elongate element 224. In some embodiments, the gripping features 226 have other shapes such as, but not limited to, frusto conical, cylindrical, cubical, ellipsoidal, and the like, or a combination thereof. The gripping features 226 may be off center in relation to the elongate element 224 in some embodiments.

In some embodiments, the gripping features 226 can comprise metallic (e.g., stainless steels, alloy steels, nitinol, brass, etc.) or polymeric materials, or a combination thereof. In some embodiments, the gripping features 226 are overmolded onto the elongate element 224. In some embodiments, the gripping features 226 are formed with the elongate element 224 as a unitary construct. In some embodiments, the gripping features 226 are attached onto the elongate element 224 after separately forming the gripping features 226 and the elongate element 224.

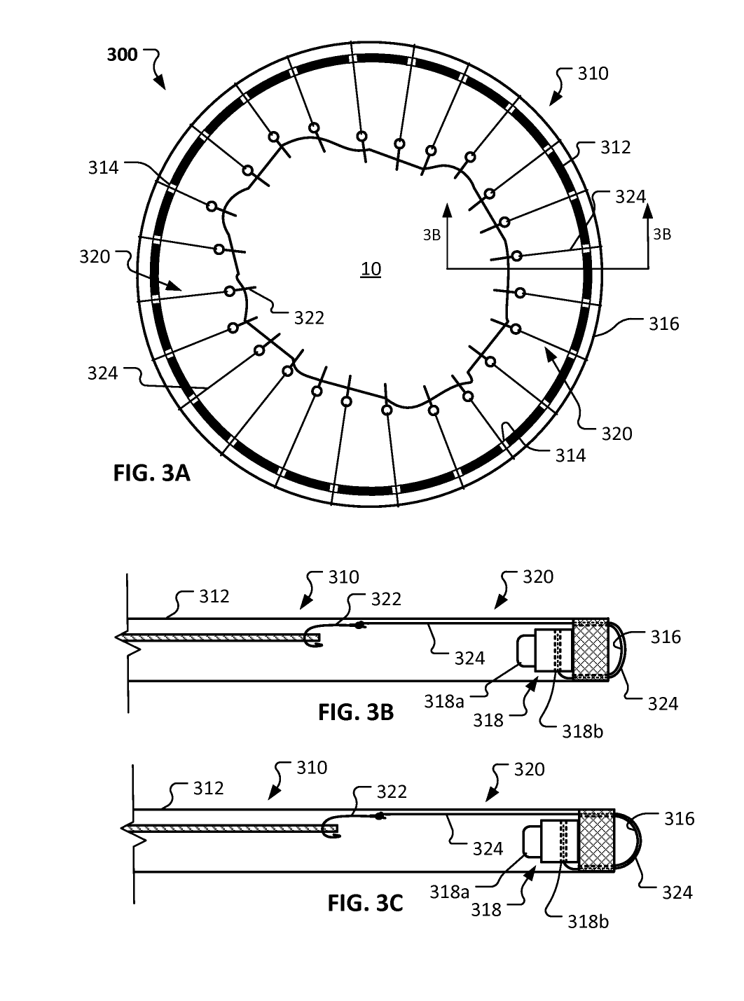

Referring to FIGS. 3A, 3B, and 3C, another example tissue tensioning apparatus 300 uses a frame 310 and multiple tensioning members 320 to exert tensile forces to a tissue 10. The multiple tensioning members 320 suspend the tissue 10 within an inner space defined by the frame 310. The frame 310 can be expanded to apply tension to the multiple tensioning members 320, which in turn apply tension to the tissue 10.

In the depicted embodiment, the multiple tensioning members 320 each include a hook 322 and an elongate element 324. The hook 322 is coupled to one end of the elongate element 324. The other end of the elongate element 324 is releasably coupled to the frame 310 as described below.

In the depicted embodiment, the frame 310 includes a circular peripheral element 312 and an inflatable member 316. In some embodiments, the frame 310 can have other shapes such as, but not limited to, rectangular, triangular, elliptical, and the like. In some embodiments, the diameter of the peripheral element 312 is about 300 mm to about 350 mm. However, peripheral elements 312 having sizes smaller than 300 mm or larger than 350 mm in diameter are also envisioned within the scope of this disclosure.

The inflatable member 316 is coupled to the peripheral element 312 in an arrangement such that the inflatable member 316 can be inflated and deflated to increase and decrease the outer diameter of the frame 310. In that sense, the inflatable member 316 acts as a frame size adjustment feature. In some embodiments, the peripheral element 312 includes multiple elongate element engagement features 314 (e.g., slots) that are spaced apart from each other at positions on the peripheral element 312 and that releasably receive the elongate elements 324.

In the depicted embodiment, one end of each of the multiple tensioning members 320 is releasably coupled to the tissue 10 using the hook 322. The elongate element 324 of each tensioning member 320 is coupled to the hook 322. The elongate element 324 of each tensioning member 320 is also releasably engaged with a spring clamp 318 affixed to the peripheral element 312 of the frame 310 in a location such that the elongate element 324 is wrapped over the inflatable member 316. In this arrangement, the multiple tensioning members 320 can suspend the tissue 10 within an inner space defined by the frame 310, and the inflatable member 316 can be inflated to increase the tension of the tensioning members 320 and tissue 10.

With the tissue 10 suspended within an inner space defined by the frame 310, the inflatable member 316 can be inflated to increase and/or deflated to decrease the diameter of the frame 310. Increasing or decreasing the diameter of the frame 310 adjusts the tension of the multiple tensioning members 320 and the tissue 10. In particular, increasing the diameter of the frame 310 increases the tension of the multiple tensioning members 320 and the tissue 10, while decreasing the diameter of the frame 310 decreases the tension of the multiple tensioning members 320 and the tissue 10. Using this arrangement, the tension of individual tensioning members 320 are controlled and maintained substantially equal to each other.

In some embodiments, the peripheral element 312 of the frame 310 is metallic or at least partially metallic. For example, in some embodiments the peripheral element 312 comprises stainless steel (e.g., L605, 304, 304L, 316, etc.), alloy steels, aluminum, or aluminum alloys to provide a few examples. In some embodiments, the peripheral element 312 of the frame 310 is polymeric or at least partially polymeric. For example, in some embodiments the peripheral element 312 comprises polyamide, polyethylene, polytetrafluoroethylene, or polyvinyl chloride to provide a few examples. In some embodiments, the peripheral element 312 comprises a metallic portion and a polymeric portion. For example, in some such embodiments the elongate element engagement features 314 may comprise a polymeric material while other portions of the frame 310 comprise one or more metallic materials.

The frame 310 also includes the inflatable member 316. The inflatable member 316 is configured to expand and retract the diameter of the frame 310 by inflation and deflation of the inflatable member 316 respectively. Using this arrangement, the tension of individual tensioning members 320 are controlled substantially equal to each other. In the depicted embodiment, the inflatable member 316 comprises a balloon surrounding substantially the entire outer diameter of the peripheral element 312. In FIG. 3B the inflatable member 316 is shown in a deflated configuration, while in FIG. 3C the inflatable member 316 is shown in an inflated configuration. Because the elongate element 324 is wrapped over the inflatable member 316, tension is applied to the elongate element 324 as the inflatable member 316 is inflated. In some embodiments, the inflatable member 316 may be located on other areas of the peripheral element 312 without departing from the scope of this disclosure.

In some embodiments, the inflatable member 316 comprises materials such as, but not limited to, PET, nylon, Kevlar, polyurethane, Pebax®, and the like. In some embodiments, the pressure of the inflation media supplied to the inflatable member 316 is proportional to the tension applied to the tissue 10. Therefore, in some such embodiments a predetermined amount of inflation media pressure may be supplied to the inflatable member 316 to result in a desired tensioning of the tissue 10.

The tissue tensioning apparatus 300 also includes the multiple tensioning members 320. The multiple tensioning members 320 each include the hook 322 and the elongate element 324. In the depicted embodiment, the hook 322 is J-shaped hook that is similar to a fishing hook. In some embodiments, other types of devices and/or other techniques for coupling the tensioning members 320 to the tissue 10 are used. For example, in some embodiments, clips, sutures, barbs, adhesives, clamps, and the like, and combinations thereof can be used to couple the tensioning members 320 to the tissue 10 are used.

Each of the multiple tensioning members 320 also includes the elongate element 324. In some embodiments, the elongate element 324 is a filament, a wire, a string, a shaft, a cord, a chain, and the like, and/or combinations thereof. The elongate element 324 can comprise metallic (e.g., stainless steels, alloy steels, nitinol, brass, etc.) or polymeric materials, or a combination thereof. In some embodiments, the elongate element 324 is flexible, but flexibility is not a requirement. In some embodiments, the elongate element 324 does not appreciably stretch when tension is applied to the elongate element 324 by the expansion of the frame 310. Alternatively, in some embodiments the elongate element 324 may stretch when so tensioned.

The elongate element 324 extends radially outward from the hook 322 towards the peripheral member 312. The elongate element 324 is wrapped over (or otherwise engaged with) the inflatable member 316. The elongate element 324 is coupled to the frame 310. In the depicted embodiment, the elongate element 324 is coupled to the frame 310 using the spring clamp 318. The spring clamp 318 can be conveniently used by depressing a spring clamp button 318a, extending the elongate element 324 into an opening 318b while maintaining the button 318a in the depressed orientation, and then releasing the button 318a to cause the elongate element 324 to be clamped within the spring clamp 318. The use of the spring clamp 318 for releasably coupling the elongate element 324 to the frame 310 is just one type of engagement feature that can be used. In some embodiments, the elongate element 324 can be coupled to the frame 310 using other types of clamps, screws, eyelets, receptacles, collets, wedges, cleats, chocks, and various other types of engagement features, and combinations thereof.

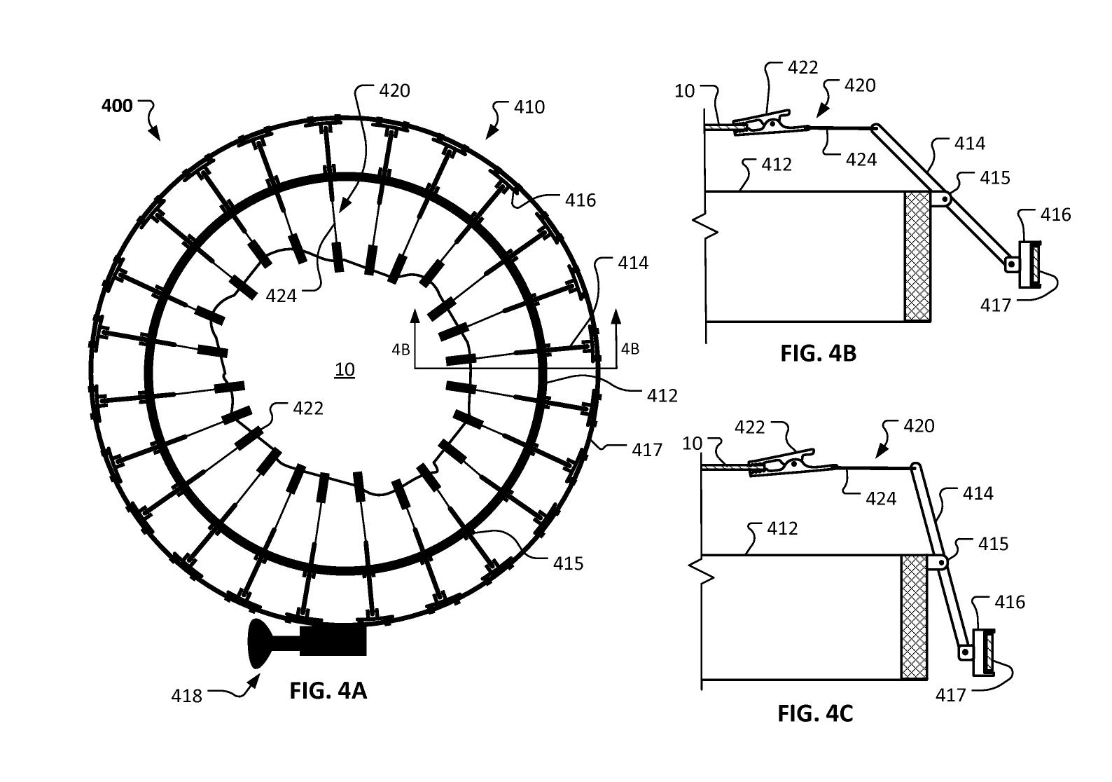

Referring to FIGS. 4A, 4B, and 4C, an example tissue tensioning apparatus 400 uses a frame 410 and multiple tensioning members 420 to exert tensile forces to a tissue 10. The multiple tensioning members 420 suspend the tissue 10 within an inner space defined by the frame 410. The frame 410 can be adjusted to apply tension to the multiple tensioning members 420, which in turn apply tension to the tissue 10.

In the depicted embodiment, the frame 410 includes a circular peripheral element 412. In some embodiments, the frame 410 can have other shapes such as, but not limited to, rectangular, triangular, elliptical, and the like. In some embodiments, the diameter of the peripheral element 412 is about 300 mm to about 350 mm.

However, peripheral elements 412 having sizes smaller than 300 mm or larger than 350 mm in diameter are also envisioned within the scope of this disclosure.

In the depicted embodiment, the multiple tensioning members 420 each include a clip 422 and an elongate element 424. Each of the multiple tensioning members 420 is releasably coupled to the tissue 10 using the clip 422. The clip 422 is coupled to one end of the elongate element 424. The other end of the elongate element 424 is coupled to a pivot arm 414. The pivot arm 414 is pivotably coupled to a clevis 415 that is fixedly attached to the peripheral element 412. The pivot arm 414 is also pivotably coupled to a peripheral band receiver 416. The location where the elongate element 424 is coupled to the pivot arm 414 is on the opposite side of the clevis 415 from the peripheral band receiver 416. Therefore, as the peripheral band receiver 416 moves radially inward, tension is applied to the elongate element 424 by the pivot arm 414.

A peripheral band 417 is slidably engaged with each peripheral band receiver 416. The diameter of the peripheral band 417 is adjustable using a peripheral band adjustment mechanism 418. The peripheral band adjustment mechanism 418 is configured to expand and retract the diameter defined by the positions of the peripheral band receivers 416 by manipulation of the peripheral band adjustment mechanism 418. FIG. 4C shows an arrangement in which the peripheral band 417 has been diametrically contracted to apply tension to the tissue 10 as compared to FIG. 4B. Using this arrangement, the tension of individual tensioning members 420 are controlled substantially equal to each other.

In the depicted embodiment, the peripheral band adjustment mechanism 418 comprises a screw or worm gear that meshes with complementary features on the peripheral band 417 to establish a worm gear and worm wheel type of arrangement. The peripheral band 417 and peripheral band adjustment mechanism 418 cooperate to increase or decrease the diameter of the peripheral band 417 like the manner of a hose clamp device. In that sense, the peripheral band 417 and the peripheral band adjustment mechanism 418 act as a frame size adjustment feature. In some embodiments, the peripheral band adjustment mechanism 418 may take other forms that serve to expand and retract the diameter of the frame 410 without departing from the scope of this disclosure.

In some embodiments, the components of the frame 410 are metallic or at least partially metallic. For example, in some embodiments the components of the frame 410 comprise stainless steel (e.g., L605, 304, 304L, 316, etc.), alloy steels, brass, aluminum, or aluminum alloys to provide a few examples. In some embodiments, the components of the frame 410 are polymeric or at least partially polymeric. For example, in some embodiments the components of the frame 410 comprise polyamide, polyethylene, polytetrafluoroethylene, or polyvinyl chloride to provide a few examples.

In some embodiments, the torque to adjust the peripheral band adjustment mechanism 418 is proportional to the tension applied to the tissue 10. Therefore, in some such embodiments a predetermined amount of torque may be applied to the peripheral band adjustment mechanism 418 to result in a desired tensioning of the tissue 10. In some implementations, a torque measuring instrument may be used to apply the torque and to thereby attain a desired amount tension on the tissue 10.

The tissue tensioning apparatus 400 also includes the multiple tensioning members 420. The multiple tensioning members 420 each include the clip 422 and the elongate element 424.

In the depicted embodiment, the clip 422 is an alligator clip 422. In some embodiments, other types of devices and/or other techniques for coupling the tensioning members 420 to the tissue 10 are used. For example, in some embodiments, hooks, sutures, barbs, adhesives, clamps, and the like, and combinations thereof can be used to couple the tensioning members 420 to the tissue 10 are used.

Each of the multiple tensioning members 420 also includes the elongate element 424. In some embodiments, the elongate element 424 is a filament, a wire, a string, a shaft, a chain, and the like, and/or combinations thereof. The elongate element 424 can comprise metallic (e.g., stainless steels, alloy steels, nitinol, brass, etc.) or polymeric materials, or a combination thereof. In some embodiments, the elongate element 424 is flexible, but flexibility is not a requirement. In some embodiments, the elongate element 424 does not appreciably stretch when tension is applied to the elongate element 424 by the adjustment of the frame 410.

Alternatively, in some embodiments the elongate element 424 may stretch when so tensioned.

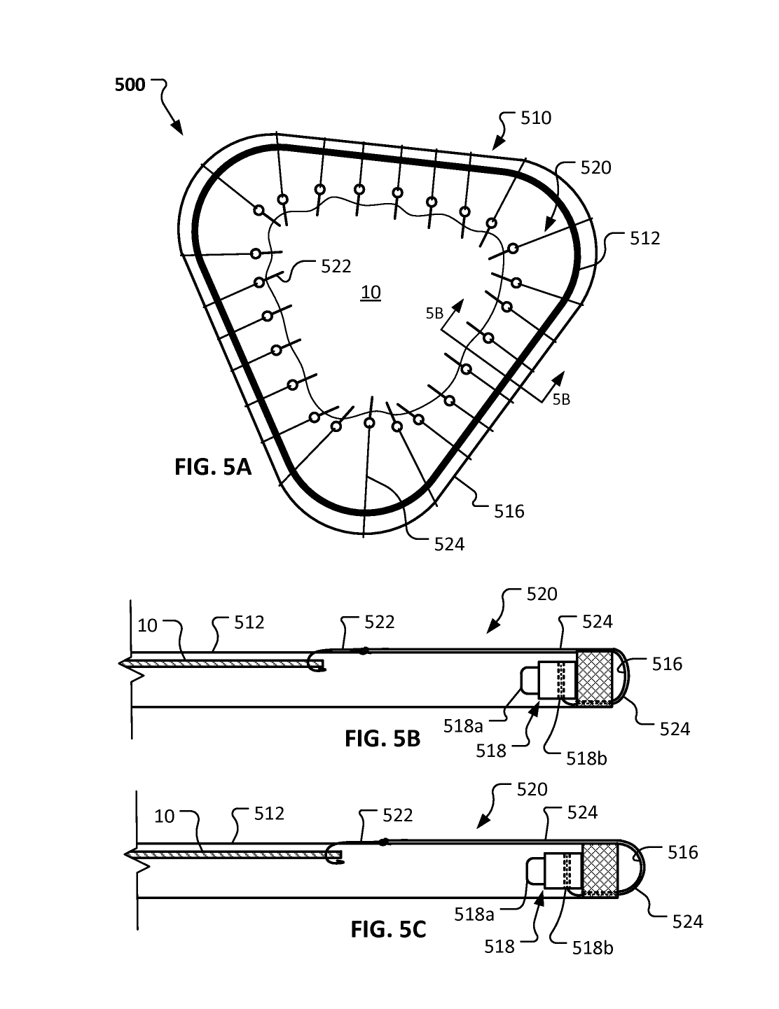

Referring to FIGS. 5A, 5B, and 5C, another example tissue tensioning apparatus 500 uses a frame 510 and multiple tensioning members 520 to exert tensile forces to a tissue 10. The multiple tensioning members 520 suspend the tissue 10 within an inner space defined by the frame 510. The frame 510 can be expanded to apply tension to the multiple tensioning members 520, which in turn apply tension to the tissue 10.

In the depicted embodiment, the multiple tensioning members 520 each include a hook 522 and an elongate element 524. The hook 522 is coupled to one end of the elongate element 524. The other end of the elongate element 524 is releasably coupled to the frame 510 as described below.

In the depicted embodiment, the frame 510 includes a triangular peripheral element 512 and an inflatable member 516. In some embodiments, the frame 510 can have other shapes such as, but not limited to, rectangular, circular, elliptical, and the like. In some embodiments, the inner open area defined by the peripheral element 512 is about 300 mm to about 350 mm wide and high. However, peripheral elements 512 having an inner open area size smaller than 300 mm or larger than 350 mm are also envisioned within the scope of this disclosure.

The inflatable member 516 is coupled to the peripheral element 512 in an arrangement such that the inflatable member 516 can be inflated and deflated to increase and decrease the outer peripheral size of the frame 510. In that sense, the inflatable member 516 acts as a frame size adjustment feature. In the depicted embodiment, one end of each of the multiple tensioning members 520 is releasably coupled to the tissue 10 using the hook 522. The elongate element 524 of each tensioning member 520 is coupled to the hook 522. The elongate element 524 of each tensioning member 520 is also releasably engaged with a spring clamp 518 affixed to the peripheral element 512 of the frame 510 in a location such that the elongate element 524 is wrapped over the inflatable member 516. In this arrangement, the multiple tensioning members 520 can suspend the tissue 10 within an inner space defined by the frame 510, and the inflatable member 516 can be inflated to increase the tension of the tensioning members 520 and tissue 10.

With the tissue 10 suspended within an inner space defined by the frame 510, the inflatable member 516 can be inflated to increase and/or deflated to decrease the outer peripheral size of the frame 510. Increasing or decreasing the outer peripheral size of the frame 510 adjusts the tension of the multiple tensioning members 520 and the tissue 10. In particular, increasing the outer peripheral size of the frame 510 increases the tension of the multiple tensioning members 520 and the tissue 10, while decreasing the outer peripheral size of the frame 510 decreases the tension of the multiple tensioning members 520 and the tissue 10. Using this arrangement, the tension of individual tensioning members 520 are controlled and maintained substantially equal to each other.

In some embodiments, the peripheral element 512 of the frame 510 is metallic or at least partially metallic. For example, in some embodiments the peripheral element 512 comprises stainless steel (e.g., L605, 304, 304L, 316, etc.), alloy steels, aluminum, or aluminum alloys to provide a few examples. In some embodiments, the peripheral element 512 of the frame 510 is polymeric or at least partially polymeric. For example, in some embodiments the peripheral element 512 comprises polyamide, polyethylene, polytetrafluoroethylene, or polyvinyl chloride to provide a few examples. In some embodiments, the peripheral element 512 comprises a metallic portion and a polymeric portion. For example, in some such embodiments the elongate element engagement features 514 may comprise a polymeric material while other portions of the frame 510 comprise one or more metallic materials. The frame 510 also includes the inflatable member 516. The inflatable member 516 is configured to expand and retract the outer peripheral size of the frame 510 by inflation and deflation of the inflatable member 516 respectively. Using this arrangement, the tension of individual tensioning members 520 are controlled substantially equal to each other. In the depicted embodiment, the inflatable member 516 comprises a balloon surrounding substantially the entire outer periphery of the peripheral element 512. In FIG. 5B the inflatable member 516 is shown in a deflated configuration, while in FIG. 5C the inflatable member 516 is shown in an inflated configuration. Because the elongate element 524 is wrapped over the inflatable member 516, tension is applied to the elongate element 524 as the inflatable member 516 is inflated. In some embodiments, the inflatable member 516 may be located on other areas of the peripheral element 512 without departing from the scope of this disclosure.

In some embodiments, the inflatable member 516 comprises materials such as, but not limited to, PET, nylon, Kevlar, polyurethane, Pebax®, and the like.

In some embodiments, the pressure of the inflation media supplied to the inflatable member 516 is proportional to the tension applied to the tissue 10. Therefore, in some such embodiments a predetermined amount of inflation media pressure may be supplied to the inflatable member 516 to result in a desired tensioning of the tissue 10.

The tissue tensioning apparatus 500 also includes the multiple tensioning members 520. The multiple tensioning members 520 each include the hook 522 and the elongate element 524. In the depicted embodiment, the hook 522 is J-shaped hook that is similar to a fishing hook. In some embodiments, other types of devices and/or other techniques for coupling the tensioning members 520 to the tissue 10 are used.

For example, in some embodiments, clips, sutures, barbs, adhesives, clamps, and the like, and combinations thereof can be used to couple the tensioning members 520 to the tissue 10 are used.

Each of the multiple tensioning members 520 also includes the elongate element 524. In some embodiments, the elongate element 524 is a filament, a wire, a string, a shaft, a cord, a chain, and the like, and/or combinations thereof. The elongate element 524 can comprise metallic (e.g., stainless steels, alloy steels, nitinol, brass, etc.) or polymeric materials, or a combination thereof. In some embodiments, the elongate element 524 is flexible, but flexibility is not a requirement. In some embodiments, the elongate element 524 does not appreciably stretch when tension is applied to the elongate element 524 by the expansion of the frame 510. Alternatively, in some embodiments the elongate element 524 may stretch when so tensioned.

The elongate element 524 extends radially outward from the hook 522 towards the peripheral member 512. The elongate element 524 is wrapped over (or otherwise engaged with) the inflatable member 516. The elongate element 524 is coupled to the frame 510. In the depicted embodiment, the elongate element 524 is coupled to the frame 510 using the spring clamp 518. The spring clamp 518 can be conveniently used by depressing a spring clamp button 518a, extending the elongate element 524 into an opening 518b while maintaining the button 518a in the depressed orientation, and then releasing the button 518a to cause the elongate element 524 to be clamped within the spring clamp 518. The use of the spring clamp 518 for releasably coupling the elongate element 524 to the frame 510 is just one type of engagement feature that can be used. In some embodiments, the elongate element 524 can be coupled to the frame 510 using other types of clamps, screws, eyelets, receptacles, collets, wedges, cleats, chocks, and various other types of engagement features, and combinations thereof.

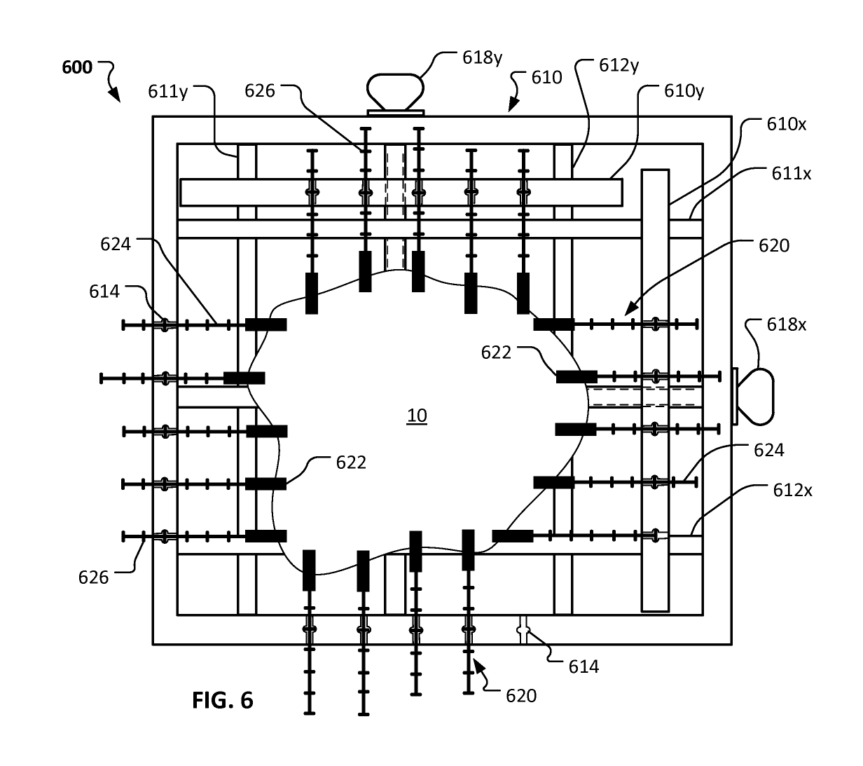

Referring to FIG. 6, an example tissue tensioning apparatus 600 uses a frame 610 and multiple tensioning members 620 to exert biaxial tensile forces to a tissue 10. The multiple tensioning members 620 suspend the tissue 10 within an inner space defined by the frame 610. The frame 610 can be adjusted to apply varying amounts of biaxial tension to the multiple tensioning members 620, which in turn apply biaxial tension to the tissue 10.

In the depicted embodiment, the frame 610 is rectangular. In some embodiments, the frame 610 can have other shapes such as, but not limited to, square, triangular, polygonal, and the like. In some embodiments, the inner width and inner height of the frame 610 is about 300 mm to about 350 mm. However, frames having an inner width and inner height that are smaller than 300 mm or larger than 350 mm are also envisioned within the scope of this disclosure.

In the depicted embodiment, the multiple tensioning members 620 each include a clip 622, an elongate element 624, and multiple gripping features 626. The clip 622 is coupled to one end of the elongate element 624. The multiple gripping features 626 are coupled to the elongate element 624 at spaced apart positions along the length of the elongate element 624. In the depicted embodiment, the elongate element 624 is a shaft and the multiple gripping features 626 are protrusions that extend laterally from the shaft. The multiple gripping features 626 are designed to be engaged with a corresponding tension member engagement feature 614 located on the frame 610. The selection of the particular gripping feature 626 to be engaged with the corresponding tension member engagement feature 614 can be determined so as to suspend the tissue 10 while imparting minimal tension thereto.

In the depicted embodiment, the clip 622 is an alligator clip 622. In some embodiments, other types of devices and/or other techniques for coupling the tensioning members 620 to the tissue 10 are used. For example, in some embodiments, hooks, sutures, barbs, adhesives, clamps, and the like, and combinations thereof can be used to couple the tensioning members 620 to the tissue 10 are used.

Each of the multiple tensioning members 620 also includes the elongate element 624, which in the depicted embodiment is a shaft. In some embodiments, the elongate element 624 is a filament, a wire, a string, a cord, a chain, and the like, and/or combinations thereof. The elongate element 624 can comprise metallic (e.g., stainless steels, alloy steels, nitinol, brass, etc.) or polymeric materials, or a combination thereof. In some embodiments, the elongate element 624 is flexible, but flexibility is not a requirement. In some embodiments, the elongate element 624 does not appreciably stretch when tension is applied to the elongate element 624 by the adjustment of the frame 610. Alternatively, in some embodiments the elongate element 624 may stretch when so tensioned.

In some embodiments, the gripping features 626 are rigidly affixed to the elongate element 624. In some embodiments, the multiple gripping features 626 are movably fixable to the elongate element 624 (e.g., using a set screw, twist lock, compression fit, collet, clamp arrangement, etc.). In some embodiments, the gripping features 626 are equally spaced apart from each other at locations along the elongate element 624. In some embodiments, some of the spacing between the gripping features 626 varies or is variable along the elongate element 624 (e.g., using graduated spacing, etc.).

In the depicted embodiment, the gripping features 626 are cylindrical and on center with the elongate element 624. In some embodiments, the gripping features 626 have other shapes such as, but not limited to, frusto conical, spherical, cubical, ellipsoidal, and the like, or a combination thereof. The gripping features 626 may be off center in relation to the elongate element 624 in some embodiments.

The frame 610 includes an x-axis adjustable member 610x and a y-axis adjustable member 610y. The adjustable member 610x can be translated in the x-axis by manipulating a lead screw 618x. The threads of the lead screw 618x mate with internal threads of the x-axis adjustable member 610x so that as the lead screw 618x is turned the x-axis adjustable member 610x translates inward toward the tissue 10 or outward away from the tissue 10. In that manner, the tension of the tissue 10 in the x-axis direction can be adjusted. Linear guide shafts 611x and 612x can be included to keep the adjustable member 610x properly oriented as it is being translated. The adjustable member 610y can be translated in the y-axis by manipulating a lead screw 618y. The threads of the lead screw 618y mate with internal threads of the y-axis adjustable member 610y so that as the lead screw 618y is turned the y-axis adjustable member 610y translates inward toward the tissue 10 or outward away from the tissue 10. In that manner, the tension of the tissue 10 in the y-axis direction can be adjusted. Linear guide shafts 611y and 612y can be included to keep the adjustable member 610y properly oriented as it is being translated. The frame 610 therefore includes a frame size adjustment feature comprising the x-axis adjustable member 610x and the y-axis adjustable member 610y.

In some embodiments, the torque to adjust the position of the adjustable member 610x by manipulating a lead screw 618x, and to adjust the position of the adjustable member 610y by manipulating a lead screw 618y, is proportional to the resulting tension applied to the tissue 10. Therefore, in some such embodiments a predetermined amount of torque may be applied to the lead screw 618x and/or the lead screw 618y to result in a desired tensioning of the tissue 10. In some implementations, a torque measuring instrument may be used to apply the torque and to thereby attain a desired amount tension on the tissue 10.

In some embodiments, the components of the frame 610 are metallic or at least partially metallic. For example, in some embodiments the components of the frame 410 comprise stainless steel (e.g., L605, 304, 304L, 316, etc.), alloy steels, brass, aluminum, or aluminum alloys to provide a few examples. In some embodiments, the components of the frame 610 are polymeric or at least partially polymeric. For example, in some embodiments the components of the frame 610 comprise polyamide, polyethylene, polytetrafluoroethylene, or polyvinyl chloride to provide a few examples.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. A tissue tensioning apparatus for tensioning a tissue, the apparatus comprising:
a peripheral frame defining an interior space;
a plurality of elongate tensioning members each including a tissue attachment member configured to attach to the tissue, each of the plurality of tensioning members being attachable to the frame such that the tissue attachment member is disposed within the interior space; and
a frame size adjustment feature coupled to the frame and arranged to adjust a configuration of the frame and positions of the tissue attachment members relative to the interior space when the plurality of tensioning members are attached to the frame such that the tissue attachment members are disposed within the interior space.

2. The apparatus of claim 1, wherein the interior space is circular.

3. The apparatus of claim 1, wherein the interior space is generally triangular.

4. The apparatus of claim 1, wherein the interior space is polygonal.

5. The apparatus of claim 1, wherein the tissue attachment member comprises a clip.

6. The apparatus of claim 1, wherein the tissue attachment member comprises a hook.

7. The apparatus of claim 1, wherein the frame size adjustment feature comprises a rotatable screw or worm gear that meshes with features on the frame.

8. The apparatus of claim 1, wherein the frame size adjustment feature comprises an inflatable balloon member.

9. The apparatus of claim 1, wherein the frame size adjustment feature comprises a pivot arm.

10. The apparatus of claim 1, wherein the frame size adjustment feature is configured to bi-axially adjust the positions of the tissue attachment members relative to the interior space.

11. The apparatus of claim 1, wherein the frame size adjustment feature is configured to adjust the positions of the tissue attachment members relative to the interior space in four or more different directions.

12. The apparatus of claim 1, wherein the frame size adjustment feature is configured to adjust the positions of the tissue attachment members relative to the interior space in eight or more different directions.

13. The apparatus of claim 1, wherein the frame size adjustment feature is configured to adjust the positions of the tissue attachment members relative to the interior space in twelve or more different directions.

14. The apparatus of claim 1, wherein the frame includes a plurality attachment features whereby the plurality of elongate tensioning members are attachable to the frame.

15. The apparatus of claim 14, wherein the plurality attachment features comprise slots.

16. The apparatus of claim 14, wherein the plurality attachment features comprise clamps.

17. A method for tissue modification, the method comprising:
attaching a tissue to a tissue tensioning apparatus, the tissue tensioning apparatus comprising:
a peripheral frame defining an interior space;
a plurality of elongate tensioning members each including a tissue attachment member configured to attach to the tissue, each of the plurality of tensioning members being attachable to the frame such that the tissue attachment member is disposed within the interior space; and
a frame size adjustment feature coupled to the frame and arranged to adjust a configuration of the frame and positions of the tissue attachment members relative to the interior space when the plurality of tensioning members are attached to the frame such that the tissue attachment members are disposed within the interior space; and
adjusting the frame size adjustment feature to apply tension to the tissue.

18. The method of claim 17, wherein an entirety of the tissue is within the interior space when the tissue is attached to the tissue tensioning apparatus.

19. The method of claim 17, further comprising applying a fixative to the tissue, wherein the fixative comprises glutaraldehyde.

20. The method of claim 17, wherein the tension applied to the tissue is about 0.1 N to about 2 N, or wherein a tensile stress applied to the tissue by the tension is about 0.01 N/mm² to about 2 N/mm².
